(19) United States
(12) Patent Application Publication
(10) Pub. No.: US 2021/0210201 A1
(43) Pub. Date: Jul. 8, 2021

(54) PRELIMINARY CLEANING MANAGEMENT APPARATUS AND PRELIMINARY CLEANING MANAGEMENT METHOD

(71) Applicant: OLYMPUS CORPORATION, Tokyo (JP)

(72) Inventor: Yoshihide ENDO, Tokyo (JP)

(73) Assignee: OLYMPUS CORPORATION, Tokyo (JP)

(21) Appl. No.: 17/205,420

(22) Filed: Mar. 18, 2021

Related U.S. Application Data
(63) Continuation of application No. PCT/JP2018/035053, filed on Sep. 21, 2018.

(51) Int. Cl. G16H 40/40; G06F 3/06; B08B 1/00; B08B 3/04; A61B 1/00

(52) U.S. Cl. CPC G16H 40/40; G06F 3/0604; G06F 3/0659; A61B 2090/701; B08B 1/002; B08B 3/04; A61B 1/00059; G06F 3/0673

(57) ABSTRACT

A memory stores identification information on a subject related to preliminary cleaning acquired by an ID acquirer a non-contact manner. A registration processing unit determines whether or not to register preliminary cleaning execution information in a server apparatus based on the identification information stored in the memory. The registration processing unit determines, when identification information on an endoscope and identification information on a cleaning operator are stored in the memory, to register preliminary cleaning execution information including the identification information on the endoscope and the identification information on the cleaning operator.

### CROSS-REFERENCE TO RELATED APPLICATIONS

This application is based upon and claims the benefit of priority from International Application No. PCT/JP2018/035053, filed on Sep. 21, 2018, the entire contents of which are incorporated herein by reference.

### BACKGROUND OF THE INVENTION

### 1. Field of the Invention

The present disclosure relates to a technology for managing preliminary cleaning of endoscopes.

### 2. Description of the Related Art

In Japan, the Japan Gastroenterological Endoscopy Society published “Guidelines for Cleaning and Disinfecting Gastroenterological Endoscopy Equipment” in 1998, and in medical facilities, cleaning and disinfection are carried out in accordance with the published guidelines to prevent infection caused by endoscopic examination. In order to efficiently perform cleaning and disinfection of endoscopes, endoscope cleaning apparatuses that automatically perform cleaning, disinfection, and rinsing are used in many medical facilities.

High-action disinfectants are used in endoscope cleaning apparatuses. However, protein residues such as mucus and blood of patients reduce the effectiveness of high-action disinfectants. Therefore, the cleaning operator need to perform a manual preliminary cleaning to remove protein residues before the main cleaning by an endoscope cleaning apparatus.

Patent Document 1 discloses a cleaning management apparatus that determines whether or not preliminary cleaning history related to a preliminary cleaning paired with a main cleaning is stored in a storage means, and if it is determined that the preliminary cleaning history is not stored, gives a warning indicating that the preliminary cleaning has not been performed.

[Patent Document 1] Japanese Patent Application Publication NO. 2012-239536

In order to ensure the traceability of endoscopes, examination execution information and cleaning execution information need to be managed in association with each other. In particular, since a preliminary cleaning is performed manually by a cleaning operator, execution information including information regarding the cleaning operator in charge needs to be registered in a management server as the cleaning history of the preliminary cleaning.

The cleaning operator performs the preliminary cleaning of endoscopes used in examinations with gloved hands. Since the gloves after the preliminary cleaning are not clean, a user interface for manually entering preliminary cleaning execution information is not preferred. Therefore, there is a strong need for a mechanism that allows a cleaning operator to easily enter preliminary cleaning execution information without touching a user interface such as a touch panel.

### SUMMARY OF THE INVENTION

In this background, a purpose of the present disclosure is to provide a technology that allows a cleaning operator to easily enter preliminary cleaning execution information.

A preliminary cleaning management apparatus according to one embodiment of the present disclosure includes a memory that stores identification information on a subject related to preliminary cleaning acquired by an ID acquirer in a non-contact manner in a memory; and a registration processing unit that determines, when the identification information on the endoscope is stored in the memory, to register preliminary cleaning execution information including the identification information on the endoscope. The registration processing unit has a subject identification unit that identifies a subject identified by the identification information stored in the memory; and a screen generation unit that displays information related to the subject identified by the subject identification unit on a display. When the subject identification unit identifies that the identification information stored in the memory is identification information on an endoscope, the screen generation unit displays information related to the endoscope and information indicating that the preliminary cleaning execution information is to be registered on a display.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram illustrating the configuration of an information management system according to an embodiment;

FIG. 2 is a diagram illustrating functional blocks of a preliminary cleaning management apparatus;

FIG. 3 is a diagram illustrating an example of a display screen of an output apparatus;

FIG. 4 is a diagram illustrating a state where a scope ID is displayed in a scope ID column;

FIG. 5 is a diagram illustrating a state where a user ID is displayed in a user ID column;

FIG. 6 is a diagram illustrating an example of a pop-up screen displayed on a screen;

FIG. 7 is a diagram illustrating an example of a display screen of the output apparatus; and

FIG. 8 is a diagram illustrating functional blocks of a management server.

### DETAILED DESCRIPTION OF THE INVENTION

The invention will now be described by reference to the preferred embodiments. This does not intend to limit the scope of the present invention, but to exemplify the invention.

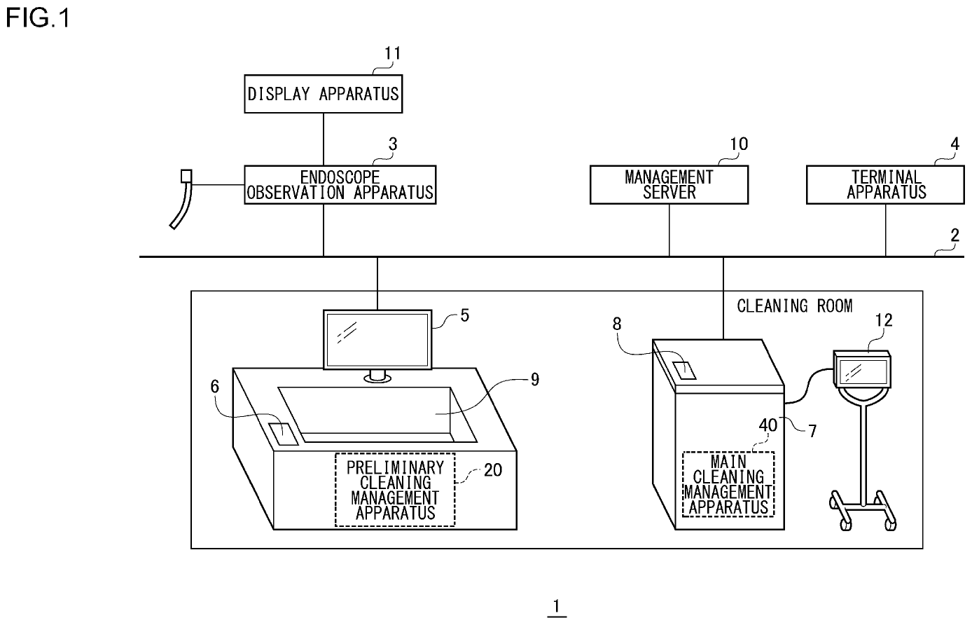

FIG. 1 illustrates the configuration of an information management system 1 according to an embodiment of the present disclosure. The information management system 1 is a system for managing endoscopes in a medical facility or the like and includes an endoscope observation apparatus 3, a terminal apparatus 4, a management server 10, and a display apparatus 11. The endoscope observation apparatus 3 and the display apparatus 11 are arranged in an examination room, and during an examination, an endoscope is connected to the endoscope observation apparatus 3, and an image captured by the endoscope is displayed on the display apparatus 11. The terminal apparatus 4 is located at a nurse station and is operated by a medical worker.

The management server 10 has a database (endoscope DB) in which information on endoscopes used in medical facilities is registered and a database (user DB) in which user information of medical workers such as doctors and cleaning operators is registered. In the endoscope DB, the type and model of an endoscope, the name given in the facility, and the like are recorded in association with identification information (scope ID) for identifying the endoscope. Further, in the user DB, information such as occupation (distinguishing between doctors, workers, or the like), name, age, sex, etc., is recorded in association with identification information (user ID) for identifying the medical worker.

The information management system 1 includes a preliminary cleaning management apparatus 20 for managing a preliminary cleaning and a main cleaning management apparatus 40 for managing the main cleaning in a cleaning room. In the embodiment, the preliminary cleaning is a cleaning that must be performed before the main cleaning by a cleaning apparatus 7 and is manually performed by a cleaning operator. The endoscope observation apparatus 3, the terminal apparatus 4, the preliminary cleaning management apparatus 20, and the main cleaning management apparatus 40 are communicably connected to the management server 10 by a network 2 such as a local area network (LAN). The management server 10 manages examination execution information and cleaning execution information related to the endoscope based on information transmitted from the endoscope observation apparatus 3, the preliminary cleaning management apparatus 20, and the main cleaning management apparatus 40.

The preliminary cleaning management apparatus 20 is connected to an ID acquirer 6 and an output apparatus 5. For example, the preliminary cleaning management apparatus 20 may be a tablet apparatus equipped with the output apparatus 5 and connected to the ID acquirer 6. The main cleaning management apparatus 40 is provided inside the cleaning apparatus 7 and is connected to an ID acquirer 8 and an output apparatus 12. Although FIG. 1 shows one cleaning apparatus 7, a plurality of cleaning apparatuses 7 may be installed in the cleaning room. Further, FIG. 1 shows that the output apparatus 12 for presenting information to the user is provided separately from the cleaning apparatus 7. However, the output apparatus 12 may be a display panel that is integrated with the upper lid, the front plate, and the like of the cleaning apparatus 7. The main cleaning management apparatus 40 may be provided outside the cleaning apparatus 7 and may be, for example, a tablet device on which the output apparatus 12 is mounted and connected to the ID acquirer 8.

The ID acquirer 6 and the ID acquirer 8 acquire identification information for identifying a subject in a non-contact manner. In the embodiment, identification information on the subject is recorded in an RFID tag, and the ID acquirer 6 and the ID acquirer 8 are RFID readers that acquire the identification information recorded in the RFID tag by short-range wireless communication. The ID acquirer 6 acquires the identification information on subjects related to a preliminary cleaning in a non-contact manner after the preliminary cleaning by a cleaning operator is completed. The subjects related to the preliminary cleaning are the subjects to be registered as the execution information of the preliminary cleaning and include an endoscope on which the preliminary cleaning has been performed and a cleaning operator who has performed the preliminary cleaning. The ID acquirer 8 acquires the identification information on subjects related to the main cleaning in a non-contact manner before the start of the main cleaning. The subjects related to the main cleaning are subjects to be registered as the execution information of the main cleaning, include at least an endoscope on which the main cleaning is performed, and may include a cleaning operator who sets the endoscope in the cleaning apparatus 7.

When an endoscope is connected to the endoscope observation apparatus 3, the endoscope observation apparatus 3 acquires identification information (scope ID) for identifying the endoscope and transmits the scope ID and information related to the examination to the management server 10. The information related to the examination includes at least examination type information, doctor-in-charge information, and examination start time. When the examination is started, the endoscope observation apparatus 3 processes image data acquired by the endoscope and displays a captured image on the display apparatus 11. Further, the endoscope observation apparatus 3 acquires the captured image at the timing when a release switch of the endoscope is pressed and transmits the captured image to an image server (not shown).

After the examination is completed, the cleaning operator wipes the outer surface of the endoscope and performs a suction cleaning of a suction/forceps channel. This cleaning is also called a bedside cleaning. The cleaning operator puts the endoscope on which the bedside cleaning has been performed into a dedicated container and carries the dedicated container to the cleaning room.

In the cleaning room, the cleaning operator performs the preliminary cleaning on the endoscope in a sink 9 with gloved hands. The output apparatus 5 is installed near the sink 9. The output apparatus 5 may be a monitor apparatus including a display and a speaker. Alternatively, the output apparatus 5 may be a display panel and a speaker of a tablet apparatus as described above. In either case, the output apparatus 5 enables screen output and/or audio output to the cleaning operator. The output apparatus 5 according to the embodiment displays the information related to the identification information on the subjects acquired by the ID acquirer 6.

In the preliminary cleaning, the cleaning operator removes an air supply/water supply button, a suction button, a forceps plug, etc., cleans the outer surface of the endoscope with running water, and then brushes the suction/forceps channel using a dedicated channel cleaning brush. Further, the cleaning operator cleans the removed air supply/water supply button, the suction button, and the forceps plug using a brush.

When the preliminary cleaning is completed, the cleaning operator brings the RFID tag attached to the endoscope close to the ID acquirer 6 and causes the ID acquirer 6 to acquire the scope ID recorded in the RFID tag. The RFID tag may be incorporated as a chip inside the endoscope or may be tied to the endoscope with a string or the like while being housed in a waterproof container. For example, when the ID acquirer 6 reads the identification information from the RFID tag, the ID acquirer 6 may generate a sound for giving a notification of the completion of the reading. After hearing the notification sound, the cleaning operator brings the RFID tag, in which the identification information (user ID) for identifying the cleaning operator himself/herself is recorded, close to the ID acquirer 6 and causes the ID acquirer 6 to acquire the user ID.

The ID acquirer 6 supplies the acquired identification information to the preliminary cleaning management apparatus 20. Although it is described in the embodiment that the ID acquirer 6 is an RFID reader, the ID acquirer 6 may be a non-contact reader apparatus according to another aspect depending on the recording form of the identification information on subjects. For example, the ID acquirer 6 may be a reader apparatus that reads one-dimensional or two-dimensional code information in a non-contact manner or may be an image processing apparatus that acquires the identification information on the subjects from a camera image. In either case, since the ID acquirer 6 has a function of acquiring identification information in a non-contact manner, the cleaning operator can allow the scope ID and the user ID to be easily acquired by the ID acquirer 6 without operating a touch panel or the like with hands wearing dirty gloves.

The endoscope on which the preliminary cleaning has been performed is set in a cleaning tank of the cleaning apparatus 7 and is subjected to the main cleaning (mechanical cleaning). Before the start of the main cleaning, the cleaning operator causes the ID acquirer 8 to acquire the scope ID of the endoscope. The cleaning operator may also allow the ID acquirer 8 to acquire the user ID of the cleaning operator.

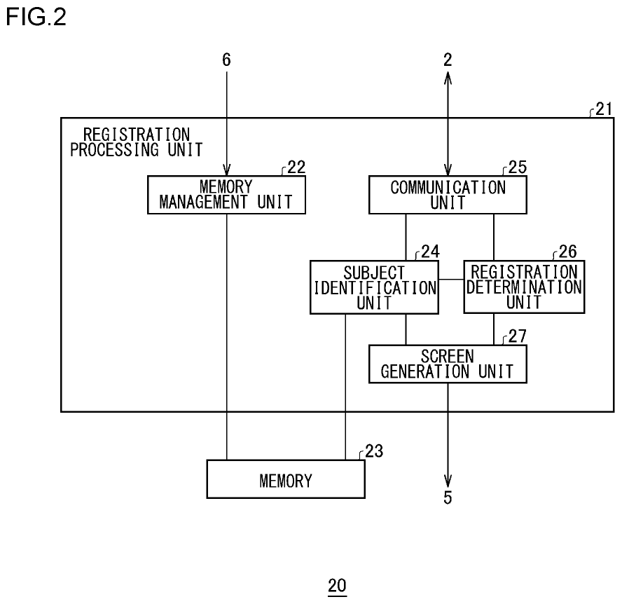

FIG. 2 illustrates functional blocks of the preliminary cleaning management apparatus 20. The preliminary cleaning management apparatus 20 includes a registration processing unit 21 and a memory 23. The memory 23 temporarily stores the identification information on the subjects related to the preliminary cleaning acquired by the ID acquirer 6 in a non-contact manner. The registration processing unit 21 determines whether or not to register preliminary cleaning execution information in the management server 10 based on the identification information stored in the memory 23. More specifically, when the scope ID and the user ID are stored in the memory 23, the registration processing unit 21 determines to register the preliminary cleaning execution information including the scope ID and the user ID in the management server 10. The registration processing unit 21 has a memory management unit 22, a subject identification unit 24, a communication unit 25, a registration determination unit 26, and a screen generation unit 27 in order to realize the registration determination function for preliminary cleaning execution information.

The configuration thereof is implemented by hardware such as an arbitrary processor, a memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

When the cleaning operator finishes the preliminary cleaning, the cleaning operator first causes the ID acquirer 6 to read one of the scope ID and the user ID and then causes the ID acquirer 6 to read the other one of the scope ID and the user ID. The cleaning operator may cause the ID acquirer 6 to read either one of the scope ID or the user ID first. However, in the following description, the scope ID is read first by the ID acquirer 6. The ID acquirer 6 supplies the identification information acquired from the RFID tag of the endoscope to the registration processing unit 21.

The memory management unit 22 manages the temporary memory of the identification information in the memory 23. When the ID acquirer 6 supplies the identification information, the memory management unit 22 stores the supplied identification information in the memory 23. At this time, the memory management unit 22 sets a timer and measures the time elapsed since the identification information is first stored in the memory 23.

The subject identification unit 24 identifies the type of the subjects identified by the identification information stored in the memory 23. The subject identification unit 24 accesses the database of the management server 10 from the communication unit 25 and acquires information (type information) that identifies the type of the subjects identified by the identification information. In this case, the subject identification unit 24 is notified by the management server 10 that the identification information supplied from the ID acquirer 6 is the information for identifying the endoscope, and the subject identification unit 24 identifies that the identification information is the scope ID of the endoscope. If the identification information includes the type information for identifying the subject type, the subject identification unit 24 may identify the subject type by referring to the type information without inquiring to the management server 10.

Subsequently, the cleaning operator causes the ID acquirer 6 to read his/her own user ID. The ID acquirer 6 supplies the identification information acquired from the RFID tag of the cleaning operator to the memory management unit 22. The memory management unit 22 stores the identification information supplied from the ID acquirer 6 in the memory 23. The subject identification unit 24 accesses the database of the management server 10 and acquires the type information of the subject identified by the identification information. In this case, the subject identification unit 24 is notified by the management server 10 that the identification information supplied from the ID acquirer 6 is the information for identifying the cleaning operator, and the subject identification unit 24 identifies that the identification information is the user ID of the cleaning operator.

The registration determination unit 26 determines to register the preliminary cleaning execution information in the management server 10 when the subjects identified by the subject identification unit 24 are an endoscope and a cleaning operator. In the preliminary cleaning management apparatus 20 according to the embodiment, the cleaning operator causing the ID acquirer 6 to read the scope ID and the user ID is the requirement for registering the preliminary cleaning execution information. In other words, by simply causing the ID acquirer 6 to read the scope ID and the user ID after the preliminary cleaning is completed, the cleaning operator can easily register the preliminary cleaning execution information in the management server 10 without requiring a manual input operation or the like. The management server 10 records the execution information of the preliminary cleaning in the preliminary cleaning history recording unit along with the reception time (or the time of transmission by the communication unit 25).

When both the scope ID and the user ID are read by the ID acquirer 6, the screen generation unit 27 preferably causes the output apparatus 5 to display information related to the subjects identified by the subject identification unit 24.

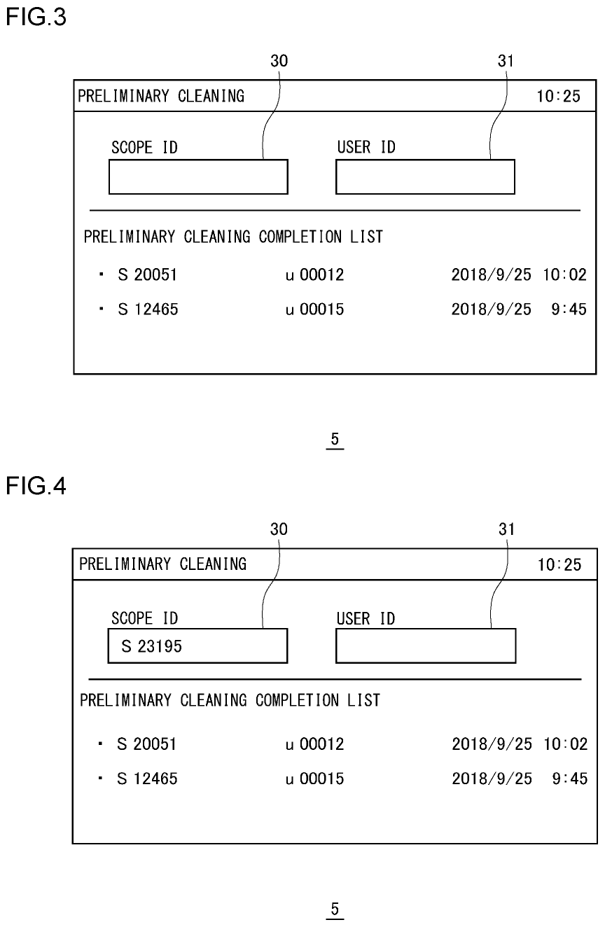

FIG. 3 illustrates an example of a display screen of the output apparatus 5. A region on the upper side of the screen is provided in order to display identification information acquired by the ID acquirer 6, that is, the identification information on an endoscope and a cleaning operator. A region on the bottom side of the screen is provided in order to display a list of preliminary cleanings performed for the day.

When the cleaning operator causes the ID acquirer 6 to read the scope ID, the memory management unit 22 stores the identification information read by the ID acquirer 6 in the memory 23. When the subject identification unit 24 identifies that the identification information stored in the memory 23 is the scope ID of the endoscope, the screen generation unit 27 displays the scope ID in a scope ID column 30.

FIG. 4 illustrates a state where a scope ID is displayed in the scope ID column 30. The scope ID displayed in the scope ID column 30 allows the cleaning operator to recognize that the scope ID of the endoscope has been appropriately acquired by the ID acquirer 6.

Subsequently, the cleaning operator causes the ID acquirer 6 to read his/her own user ID, and the memory management unit 22 stores the identification information read by the ID acquirer 6 in the memory 23. When the subject identification unit 24 identifies that the identification information stored in the memory 23 is the user ID of the cleaning operator, the screen generation unit 27 displays the user ID in a user ID column 31.

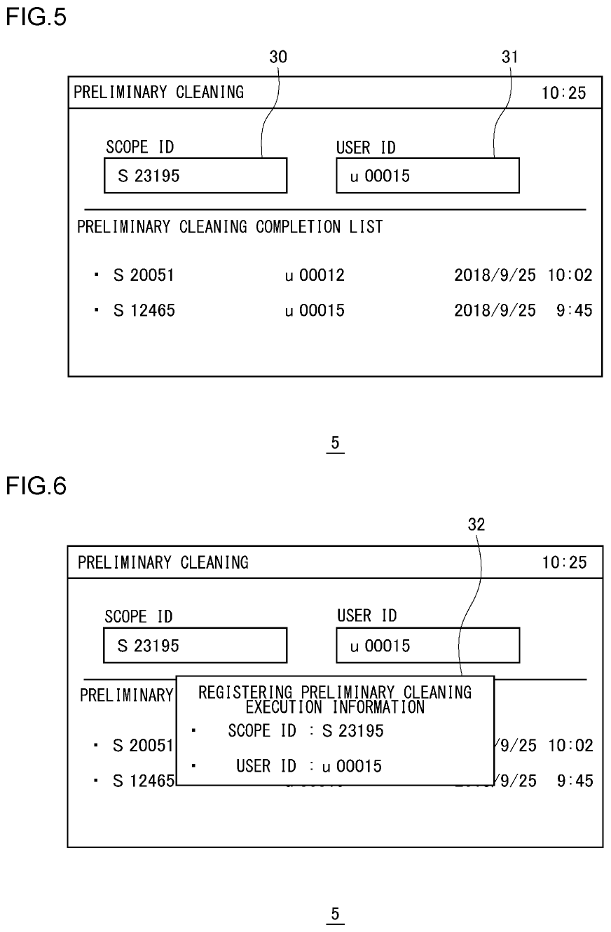

FIG. 5 illustrates a state where the user ID is displayed in the user ID column 31. The user ID displayed in the user ID column 31 allows the cleaning operator to recognize that his/her own user ID has been appropriately acquired by the ID acquirer 6.

Although identification information is shown as information related to the subjects in FIGS. 4 and 5, for example, the name of the endoscope or the name of the cleaning operator may be displayed. The subject identification unit 24 acquires the name of the endoscope from the endoscope DB and the name of the cleaning operator from the user DB, thereby the screen generation unit 27 can display the name of the endoscope and the name of the cleaning operator.

When both the scope ID and the user ID are stored in the memory 23, the registration determination unit 26 determines to register the preliminary cleaning execution information including the scope ID and the user ID in the management server 10. The subject identification unit 24 may periodically check the memory status of the memory 23 and determine whether both the scope ID and the user ID are stored. When the subject identification unit 24 determines that both the scope ID and the user ID are stored in the memory 23, the registration determination unit 26 determines that the preliminary cleaning execution information is to be registered in the management server 10. At this time, the screen generation unit 27 may display information indicating that the preliminary cleaning execution information is to be registered on the output apparatus 5.

FIG. 6 illustrates an example of a pop-up screen 32 displayed on a screen. Looking at the pop-up screen 32, the cleaning operator recognizes that the preliminary cleaning execution information is registered in the management server 10. After that, the cleaning operator carries the endoscope to the cleaning apparatus 7 and causes the ID acquirer 8 to read the scope ID before the start of the main cleaning.

As described, in the embodiment, when the subject identification unit 24 identifies either one of the subjects, the endoscope and the cleaning operator, the screen generation unit 27 causes the output apparatus 5 to display information related to the identified subject (see FIG. 4). After that, when the subject identification unit 24 identifies the other one of the subjects, the endoscope and the cleaning operator, the screen generation unit 27 displays the information related to the other one of the subjects and information indicating that the preliminary cleaning execution information is to be registered on the output apparatus 5 (see FIG. 6). The display of the pop-up screen 32 may be about several seconds. While the pop-up screen 32 is being displayed, the registration determination unit 26 transmits the preliminary cleaning execution information from the communication unit 25 to the management server 10. The management server 10 registers the preliminary cleaning execution information in the preliminary cleaning history recording unit along with time information.

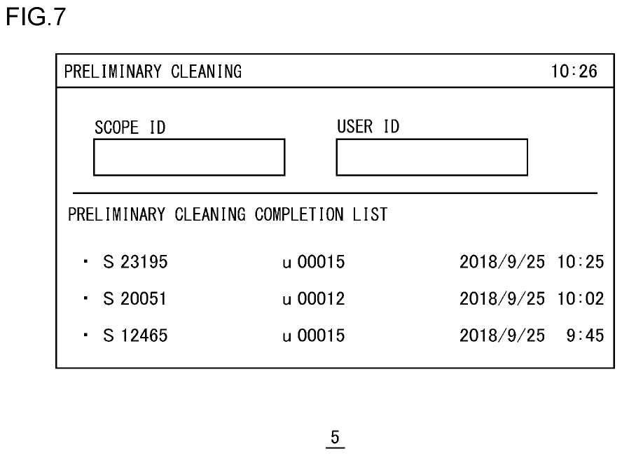

FIG. 7 illustrates an example of a display screen of the output apparatus 5. The screen generation unit 27 ends the display of the pop-up screen 32 and additionally displays the scope ID and the user ID transmitted to the management server 10 in a preliminary cleaning completion list.

When the identification information on either one of the endoscope and the cleaning operator is stored in the memory 23 and the identification information on the other one of the endoscope and the cleaning operator is then stored in the memory 23 within a predetermined period of time, the registration determination unit 26 may determine to register the preliminary cleaning execution information in the management server 10.

For example, a case is now considered where the user ID is stored in the memory 23 after 10 minutes or more have passed since the scope ID was stored in the memory 23. Normally, at the end of the preliminary cleaning, the cleaning operator causes the ID acquirer 6 to continuously read the scope ID and the user ID. Therefore, the interval for reading identification information is about 10 seconds at the longest. Therefore, if the reading interval is 10 minutes or more, it can be determined that this is for the entry of the identification information on the subjects related to two different preliminary cleanings.

From this, conversely, if the reading interval between the two pieces of identification information is short, it can be determined that this is for the entry of the identification information on the subjects related to the same preliminary cleaning. Thus, when either one of the scope ID and the user ID is stored in the memory 23 and the other one is then stored in the memory 23 within a predetermined period of time, the registration determination unit 26 determines to register the scope ID and the user ID in the management server 10 as execution information of the same preliminary cleaning. For example, the predetermined period of time may be 30 seconds.

When the identification information on either one of the endoscope and the cleaning operator is stored in the memory 23 and the identification information on the other one of the endoscope and the cleaning operator is not stored in the memory 23 within a predetermined period of time, the memory management unit 22 may delete the identification information on the one of the endoscope and the cleaning operator from the memory 23. Therefore, if the user ID is not stored in the memory 23 within a predetermined period of time (for example, 30 seconds) after the scope ID is stored in the memory 23, the memory management unit 22 deletes the stored scope ID from the memory 23. At this time, the screen generation unit 27 deletes the scope ID from the scope ID column 30. Thereby, the cleaning operator recognizes that the reading of the user ID has been delayed and causes the ID acquirer 6 to read the scope ID and the user ID again.

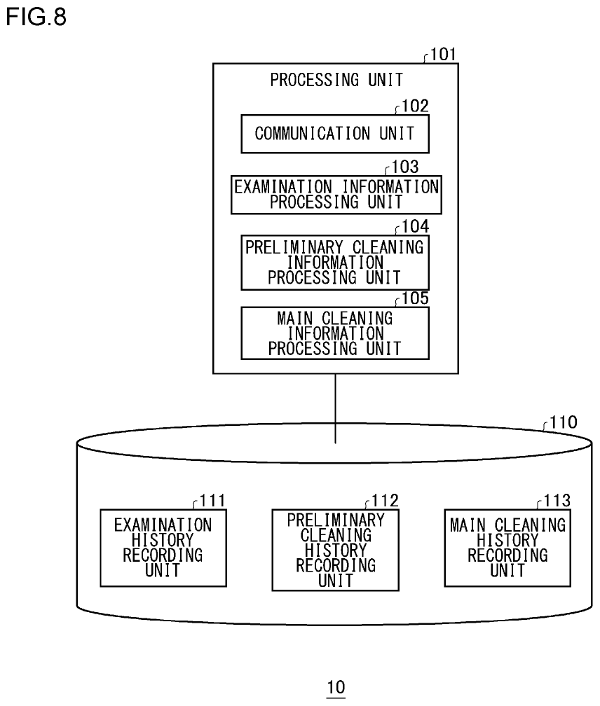

FIG. 8 illustrates functional blocks of the management server 10. The management server 10 includes a processing unit 101 and a history recording unit 110. The processing unit 101 has a communication unit 102, an examination information processing unit 103, a preliminary cleaning information processing unit 104, and a main cleaning information processing unit 105, and the history recording unit 110 has an examination history recording unit 111, a preliminary cleaning history recording unit 112, and a main cleaning history recording unit 113.

At the start of the examination, the endoscope observation apparatus 3 transmits the scope ID and information related to the examination to the management server 10. The information related to the examination includes at least examination type information, doctor-in-charge information, and examination start time. When the communication unit 102 receives the scope ID and the information related to the examination in the management server 10, the examination information processing unit 103 registers the received information in the examination history recording unit 111.

When the cleaning operator causes the ID acquirer 6 to read the scope ID and the user ID at the time of the completion of the preliminary cleaning, the preliminary cleaning management apparatus 20 transmits the preliminary cleaning execution information to the management server 10 along with reading time information. When the communication unit 102 receives the preliminary cleaning execution information in the management server 10, the preliminary cleaning information processing unit 104 registers the received information in the preliminary cleaning history recording unit 112.

When the cleaning operator causes the ID acquirer 8 to read the scope ID and the user ID before the start of the main cleaning, the main cleaning management apparatus 40 transmits the scope ID of the endoscope to be cleaned hereafter to the management server 10 along with reading time information. When the communication unit 102 receives the scope ID in the management server 10, the main cleaning information processing unit 105 searches the preliminary cleaning history recording unit 112 for the preliminary cleaning execution information of the endoscope identified by the scope ID.

The search conditions are as follows.

(Condition 1) The scope ID is the same.

(Condition 2) Not yet associated with the main cleaning.

(Condition 3) Registered in the preliminary cleaning history recording unit 112 before the date and time of the reading time information.

(Condition 4) Registered in the preliminary cleaning history recording unit 112 within 12 hours from the date and time of the reading time information.

The main cleaning information processing unit 105 identifies the execution information of the preliminary cleaning that falls under Conditions 1 to 4. When a plurality of pieces of execution information fall under Conditions 1 to 4, the main cleaning information processing unit 105 selects execution information having the latest time information. The main cleaning information processing unit 105 provides the main cleaning management apparatus 40 with the identified execution information of the preliminary cleaning. In response to this, the main cleaning management apparatus 40 causes the output apparatus 12 to display the execution information of the preliminary cleaning that makes a pair with the main cleaning. The cleaning operator can look at the display screen of the output apparatus 12 and recognize that the preliminary cleaning has been properly performed and that the cleaning information of the preliminary cleaning has been properly registered in the management server 10.

After that, the cleaning operator may enter information related to the main cleaning through an operation panel of the cleaning apparatus 7. The entered information is transmitted from the main cleaning management apparatus 40 to the management server 10 at a predetermined timing as the execution information of the main cleaning. The main cleaning information processing unit 105 registers the execution information of the main cleaning in the main cleaning history recording unit 113. At this time, the main cleaning information processing unit 105 associates the execution information of the preliminary cleaning identified before the start of the main cleaning with the execution information of the main cleaning. In this way, the management server 10 associates the execution information of the preliminary cleaning and the execution information of the main cleaning with each other. The management server 10 can ensure the traceability of the endoscope by associating the examination execution information with the cleaning execution information.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure. In the embodiments, an example is shown where the preliminary cleaning management apparatus 20 is provided in the cleaning room. However, the preliminary cleaning management apparatus 20 may be provided anywhere. Further, the preliminary cleaning management apparatus 20 may be realized as a function of the management server 10.

What is claimed is:

1. A preliminary cleaning management apparatus that manages preliminary cleaning of an endoscope, comprising:
a processor comprising hardware, wherein the processor is configured to:
store identification information on a subject related to preliminary cleaning acquired by an ID acquirer in a non-contact manner in a memory;
identify that the identification information stored in the memory is identification information on an endoscope;
when the identification information on the endoscope is stored in the memory, determine to register preliminary cleaning execution information including the identification information on the endoscope; and
display information related to the endoscope and information indicating that the preliminary cleaning execution information is to be registered on a display.

2. The preliminary cleaning management apparatus according to claim 1, wherein
the processor is configured to:
when the identification information on either one of the endoscope and a cleaning operator is stored in the memory and the identification information on the other one of the endoscope and the cleaning operator is then stored in the memory within a predetermined period of time, determine to register the preliminary cleaning execution information.

3. The preliminary cleaning management apparatus according to claim 1, wherein
the processor is configured to:
when identification information on either one of an endoscope and a cleaning operator is stored in the memory and identification information on the other one of the endoscope and the cleaning operator is not stored in the memory within a predetermined period of time, delete the identification information on the one of the endoscope and the cleaning operator from the memory.

4. The preliminary cleaning management apparatus according to claim 1, wherein
the processor is configured to:
identify that one piece of the identification information stored in the memory is identification information on either one of an endoscope and a cleaning operator;
display information related to the one of the endoscope and the cleaning operator on a display;
identify that another piece of the identification information stored in the memory is identification information on the other one of the endoscope and the cleaning operator;
display information related to the other one of the endoscope and the cleaning operator and information indicating that preliminary cleaning execution information is to be registered on a display; and
when the identification information on the endoscope and the identification information on the cleaning operator are stored in the memory, determine to register preliminary cleaning execution information including the identification information on the endoscope and the identification information on the cleaning operator.

5. The preliminary cleaning management apparatus according to claim 1, wherein
the processor is configured to:
after ending the display of the information indicating that the preliminary cleaning execution information is to be registered, display a preliminary cleaning completion list to which information related to the endoscope has been added on a display.

6. A preliminary cleaning management method for managing preliminary cleaning of an endoscope, comprising:
storing identification information on a subject related to preliminary cleaning acquired by an ID acquirer in a non-contact manner in a memory;
identifying that the identification information stored in the memory is identification information on an endoscope;
when the identification information on the endoscope is stored in the memory, determining to register preliminary cleaning execution information including the identification information on the endoscope; and
displaying information related to the endoscope and information indicating that the preliminary cleaning execution information is to be registered on a display.

7. The preliminary cleaning management method according to claim 6, wherein
determining to register preliminary cleaning execution information includes determining, when identification information on either one of an endoscope and a cleaning operator is stored in the memory and identification information on the other one of the endoscope and the cleaning operator is then stored in the memory within a predetermined period of time, to register the preliminary cleaning execution information.

8. The preliminary cleaning management method according to claim 6, further comprising:
when identification information on either one of an endoscope and a cleaning operator is stored in the memory and identification information on the other one of the endoscope and the cleaning operator is not stored in the memory within a predetermined period of time, deleting the identification information on the one of the endoscope and the cleaning operator from the memory.

9. The preliminary cleaning management method according to claim 6, comprising:
identifying that one piece of the identification information stored in the memory is identification information on either one of an endoscope and a cleaning operator;
displaying information related to the one of the endoscope and the cleaning operator on a display;
identifying that another piece of the identification information stored in the memory is identification information on the other one of the endoscope and the cleaning operator; and
displaying information related to the other one of the endoscope and the cleaning operator and information indicating that preliminary cleaning execution information is to be registered on a display, wherein
determining to register preliminary cleaning execution information includes determining, when the identification information on the endoscope and the identification information on the cleaning operator are stored in the memory, to register preliminary cleaning execution information including the identification information on the endoscope and the identification information on the cleaning operator.

10. The preliminary cleaning management method according to claim 6, further comprising:
after ending the display of the information indicating that the preliminary cleaning execution information is to be registered, displaying a preliminary cleaning completion list to which information related to the endoscope has been added on a display.
